(19) United States
(12) Patent Application Publication
(10) Pub. No.: US 2006/0020152 A1
(43) Pub. Date: Jan. 26, 2006

(54) Method for the low temperature selective oxidation of hydrogen contained in a hydrocarbon stream

(76) Inventors: Eugene Harry Theobald, Richmond, TX (US); David Morris Hamilton JR., Sugar Land, TX (US); James Allen Wambaugh, Cypress, TX (US)

Correspondence Address: SHELL OIL COMPANY, P O BOX 2463, HOUSTON, TX, 772522463, US

(21) Appl. No.: 10/896,384

(22) Filed: Jul. 22, 2004

(51) Int. Cl. C07C 4/02

(52) U.S. Cl. 585/443

(57) ABSTRACT

A method for the low temperature selective oxidation of hydrogen contained in a feed comprising hydrogen and dehydrogenatable hydrocarbons and, in particular, for selectively oxidizing the hydrogen of a dehydrogenation reactor effluent. The feed is contacted under low temperature selective oxidation reaction conditions and in the presence of oxygen with a selective oxidation catalyst that is preferably a noble metal supported on an inorganic support material. The low temperature selective oxidation reactor can be operated in combination with a dehydrogenation reactor and a compressor in a manner so as to lower the operating pressure of the dehydrogenation reactor and thereby improve its operation.

### BACKGROUND OF THE INVENTION

The invention relates to the selective oxidation of hydrogen that is contained in a hydrocarbon stream. Another aspect of the invention relates to the low temperature selective oxidation of the hydrogen of a hydrocarbon stream containing hydrogen and an oxidizable hydrocarbon by contacting such hydrocarbon stream with a selective oxidation catalyst under suitable reaction conditions.

Unsaturated hydrocarbons may be manufactured by methods that include the catalytic dehydrogenation of dehydrogenatable hydrocarbons. One such method includes the dehydrogenation of ethylbenzene by use of an iron-based catalyst to yield styrene and hydrogen. This reaction is an endothermic equilibrium reaction that is thermodynamically limited. High reaction temperature and low reaction pressure favor the forward reaction to yield styrene and hydrogen.

One of the ongoing efforts to improve the operation of styrene manufacturing processes includes the use of oxidative reheat methods. The techniques associated with such methods are designed to offset the temperature lowering effect of the endothermic ethylbenzene dehydrogenation reaction by oxidizing the hydrogen formed during the dehydrogenation reaction of the ethylbenzene and using the heat released to maintain the dehydrogenation reaction temperature.

One method that utilizes the heat released from the selective oxidation of hydrogen that is contained in a dehydrogenation reaction product is presented in U.S. Pat. No. 5,994,606. This patent discloses the selective oxidation of hydrogen that is carried out in a separate reaction zone from the dehydrogenation reaction zone. The reactor effluent from the hydrogen oxidation reaction zone is passed to a second dehydrogenation zone with heat being provided by the exothermic hydrogen oxidation reaction. The selective oxidation reaction is conducted preferably within a temperature range of from 300° C. to 800° C. A too low of an oxidation reaction temperature is not desired due to loss of activity at the lower temperature.

U.S. Pat. Nos. 4,914,249; 4,812,597; 4,717,781; 4,717,779; 4,691,071; 4,652,687; 4,565,898, and 4,435,607 disclose processes that selectively oxidize the hydrogen of a dehydrogenation reaction effluent in a separate catalytic oxidation zone. The product of the selective hydrogen oxidation step is then subjected to a dehydrogenation step. Significant in all of these processes is that interposed between multiple dehydrogenation steps is a selective oxidation step that uses a specifically defined selective oxidation catalyst that must be stable at the severe selective oxidation reaction conditions to which the catalyst is subjected. The selective oxidation reaction conditions include the contacting of the dehydrogenation reactor effluent with the oxidation catalyst at a temperature in the range of from about 600° C. to 650° C. in the presence of steam.

### SUMMARY OF THE INVENTION

It is, thus, an object of this invention to provide a new method that provides for an improved operation of a dehydrogenation reaction system.

Another object of the invention is to provide for the low temperature selective oxidation of hydrogen that is contained in a dehydrogenation reactor effluent.

Accordingly, an inventive method is provided for the low temperature selective oxidation of hydrogen contained in a reactor effluent of a dehydrogenation reactor. In this method, at least a portion of the hydrogen contained in the reactor effluent is selectively oxidized by contacting the reactor effluent under low temperature selective oxidation conditions and in the presence of oxygen with a selective oxidation catalyst that is effective in the selective oxidation of hydrogen when in the presence of an oxidatable hydrocarbon.

In another embodiment of the invention, provided is a method for the low temperature selective oxidation of hydrogen contained in a reactor effluent of a dehydrogenation reactor. This method includes the addition of an oxygen-containing gas to the reactor effluent to thereby form a selective oxidation reaction gas. The selective oxidation reaction gas is contacted under low temperature selective oxidation conditions with a selective oxidation catalyst that is effective in the selective oxidation of the hydrogen contained in the selective oxidation reaction gas to thereby yield a selectively oxidized reaction product having a reduced amount of hydrogen relative to the amount of the hydrogen in the reactor effluent.

Yet another embodiment of the inventive method includes the improvement in the operation of a dehydrogenation reactor system that is operated under dehydrogenation reaction conditions, including a dehydrogenation pressure, and from which is yielded a reactor effluent containing hydrogen. In this method a low temperature selective oxidation reactor system is provided which is operatively connected with the dehydrogenation reactor system so as to be capable of receiving the reactor effluent as a feed. The low temperature selective oxidation reactor system is operated under low temperature selective oxidation conditions so as to selectively oxidize at least a portion of the hydrogen contained in the reactor effluent and to reduce the dehydrogenation pressure of the dehydrogenation reactor system.

Still another embodiment of the inventive method includes an improvement in the operation of a dehydrogenation reactor system, which comprises a dehydrogenation reactor that defines a dehydrogenation reaction zone containing a dehydrogenation catalyst and includes a dehydrogenation reactor feed inlet for receiving a dehydrogenation reactor feed and a dehydrogenation reactor effluent outlet for discharging a dehydrogenation reactor effluent. Provided is a low temperature selective oxidation reactor system, which comprises an oxidation reactor that defines an oxidation reaction zone containing a low temperature selective oxidation catalyst and includes an oxidation reactor feed inlet for receiving the dehydrogenation reactor effluent and an oxidation reactor effluent outlet for discharging a selective oxidation reactor effluent. The dehydrogenation reactor system is operated under dehydrogenation reaction conditions, which include a first dehydrogenation pressure, so as to yield from the dehydrogenation reaction zone the dehydrogenation reactor effluent that contains hydrogen. The dehydrogenation reactor effluent is introduced into the oxidation reaction zone through the oxidation reactor feed inlet while operating the oxidation reaction zone under low temperature selective oxidation reaction conditions so as to selectively oxidize at least a portion of the hydrogen contained in the reactor effluent and to reduce the first dehydrogenation pressure of the dehydrogenation reactor system to a second dehydrogenation pressure. The selective oxidation reactor effluent is yielded from the oxidation reaction zone through the oxidation reactor effluent outlet.

Other objects and advantages of the invention will become apparent from the following detailed description and appended claims.

### BRIEF DESCRIPTION OF THE DRAWINGS

FIG. 1 is a schematic representation of a styrene manufacturing process that includes a step for the selective oxidation of hydrogen that is contained in an ethylbenzene dehydrogenation reactor effluent stream.

FIG. 2 presents plots of the percent hydrogen conversion as a function of reaction temperature achieved with various types of catalysts used in experiments involving the oxidation of hydrogen that is contained in a simulated ethylbenzene dehydrogenation reaction effluent product.

FIG. 3 presents plots of the percent styrene conversion as a function of reaction temperature corresponding to the same catalysts, feed and experimental reaction conditions as are presented in FIG. 1.

### DETAILED DESCRIPTION OF THE INVENTION

The inventive method solves some of the problems associated with certain of the known dehydrogenation processes that include a step to selectively oxidize hydrogen contained in the dehydrogenation reaction effluent stream. These processes supposedly use the heat generated by the exothermic hydrogen oxidation reaction to provide for the heat input needed for a second, endothermic dehydrogenation step and to provide for a shift in the equilibrium conditions toward the formation of the dehydrogenated compounds and hydrogen. The selective hydrogenation step of such known processes necessarily is conducted at high temperature reaction conditions that are comparable to the required dehydrogenation reaction temperatures ranging upwardly to 650° C. or greater and generally no lower than about 400° C. The inventive method, on the other hand, provides for the low temperature selective oxidation of hydrogen contained in a dehydrogenation reactor effluent stream.

Other embodiments of the invention described and claimed herein, in addition to providing for the low temperature selective oxidation of hydrogen, also provide for an improved operation of a dehydrogenation reactor system by lowering the pressure at which the dehydrogenation reactor is operated to thereby shift the equilibrium conditions therein toward the yielding of a dehydrogenated compound and hydrogen.

In one embodiment of the invention, a dehydrogenation reactor effluent undergoes a low temperature selective oxidation step in which at least a portion of the hydrogen contained in the dehydrogenation reactor effluent is selectively oxidized. The low temperature selective oxidation step is conducted by contacting the dehydrogenation reactor effluent with a low temperature selective oxidation catalyst in the presence of oxygen and under suitable low temperature selective oxidation conditions. The selective oxidation catalyst must be effective in the selective oxidation of hydrogen when in the presence of an oxidatable hydrocarbon and, thus, provide for the selective oxidation of hydrogen contained in a stream comprising an oxidatable hydrocarbon, such as, for example, styrene.

The feed that is subjected to the low temperature selective oxidation can be any feed material that comprises hydrogen and an oxidatable hydrocarbon, such as those compounds having the general formula: 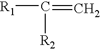
wherein R₁ and R₂ each represent an alkyl, an alkenyl or a phenyl group or a hydrogen atom. A specific example of an oxidatable hydrocarbon is styrene. The feed material can further comprise water that is preferably in the vapor state, i.e., steam. A significant portion of the feed material can generally comprise hydrogen. The hydrogen can be present in the feed material in an amount relative to the amount of oxidatable hydrocarbon in the range of from about 0.5 to about 2 moles of hydrogen per mole of oxidatable hydrocarbon. The amount of steam relative to the amount of oxidatable hydrocarbon that can be present in the feed material can be in the range of from about 0.1 to about 20 moles of steam per mole of oxidatable hydrocarbon.

The preferred feed material is a dehydrogenation reactor effluent resulting from the dehydrogenation of a dehydrogenatable hydrocarbon having the general formula: 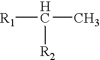
wherein R₁ and R₂ each represent an alkyl, an alkenyl or a phenyl group or a hydrogen atom. Among these, the preferred dehydrogenatable hydrocarbon is ethylbenzene.

To yield a dehydrogenation reactor effluent suitable for use as a feed material for the low temperature selective oxidation step of the invention, a dehydrogenation reactor feed comprising a dehydrogenatable hydrocarbon and, preferably, further comprising, steam, is charged to the reaction zone defined by a dehydrogenation reactor operated under dehydrogenation reaction conditions to thereby yield a dehydrogenation reactor effluent. Within the reaction zone the dehydrogenation reactor feed is contacted with a dehydrogenation catalyst.

The preferred dehydrogenation catalyst of the dehydrogenation reactor can be any known iron or iron oxide based catalyst that can suitably be used in the dehydrogenation of hydrocarbons. Such dehydrogenation catalysts include those catalysts that comprise iron oxide. The iron oxide of the dehydrogenation catalyst may be in any form and obtained from any source or by any method that provides a suitable iron oxide material for use in the iron oxide based dehydrogenation catalyst. One particularly desirable iron oxide based dehydrogenation catalyst includes potassium oxide and iron oxide.

The iron oxide of the iron oxide based dehydrogenation catalyst can be in a variety of forms including any one or more of the iron oxides, such as, for example, yellow iron oxide (goethite, FeOOH), black iron oxide (magnetite, Fe₃O₄), and red iron oxide (hematite, Fe₂O₃), or it can be combined with potassium oxide to form potassium ferrite (K₂Fe₂O₄), or it can be combined with potassium oxide to form one or more of the phases containing both iron and potassium as represented by the formula K₂.OFe₂O₃.

Typical iron based dehydrogenation catalysts comprise from 10 to 100 weight percent iron, calculated as Fe₂O₃, and up to 40 weight percent potassium, calculated as K₂O. The iron based dehydrogenation catalyst can further comprise one or more promoter metals that are usually in the form of an oxide. These promoter metals can be selected from the group consisting of Sc, Y, La, Mo, W, Ca, Mg, V, Cr, Co, Ni, Mn, Cu, Zn, Cd, Al, Sn, Bi, rare earths and mixtures of any two or more thereof. Among the promoter metals, preferred are those selected from the group consisting of Ca, Mg, Mo, W, Ce, Cr, V and mixtures of two or more thereof. Most preferred are Ca, Mg, and Ca.

The iron oxide based catalyst is prepared by any method known to those skilled in the art. The iron oxide based dehydrogenation catalyst comprising potassium oxide and iron oxide can, in general, be prepared by combining the components of an iron-containing compound and a potassium-containing compound, agglomerating these components to form particles, and calcining the particles. The promoter metal-containing compounds can also be combined with the iron-containing and potassium-containing components.

The catalyst components can be formed into particles such as extrudates, pellets, tablets, spheres, pills, saddles, trilobes, tetralobes and the like. One preferred method of making the iron based dehydrogenation catalyst is to mix together the catalyst components with water or a plasticizer, or both, and forming an extrudable paste from which extrudates are formed. The extrudates are then dried and calcined. The calcination is preferably done in an oxidizing atmosphere, such as air, and at temperatures upwardly to 1100° C., but, preferably from 500° C. to 1050° C., and, most preferably, from 700° C. to 1000° C.

The dehydrogenation conditions can include a dehydrogenation reaction temperature in the range of from about 500° C. to about 1000° C., preferably, from 525° C. to 750° C., and, most preferably, from 550° C. to 700° C. Thus, the first temperature of the dehydrogenation catalyst bed can range from about 500° C. to about 1000° C., more specifically, from 525° C. to 750° C., and, most specifically, from 550° C. to 700° C.

The dehydrogenation reaction pressure is a particularly important reaction condition in that lower reaction pressures in the dehydrogenation of ethylbenzene favor the forward reaction toward the formation of styrene and hydrogen. Thus, relatively low reaction pressures are desired for the dehydrogenation of ethylbenzene and can range from a vacuum pressure of as low as 5 or 6 psia upwardly to about 25 psia. The liquid hourly space velocity (LHSV) can be in the range of from about 0.1 hr⁻¹ to about 5 hr⁻¹. When styrene is being manufactured by the dehydrogenation of ethylbenzene, it is generally desirable to use steam as a diluent usually in a molar ratio of steam to ethylbenzene in the range of 0.1 to 20. Steam can also be used as a diluent with other dehydrogenatable hydrocarbons.

In the production of styrene, the dehydrogenation reactor effluent can comprise styrene, hydrogen and steam. The amount of hydrogen in the dehydrogenation reactor effluent relative to the amount of styrene can be in the range of from about 0.5 to about 2 moles of hydrogen per mole of styrene. More typically, the hydrogen-to-styrene molar ratio is in the range of from 0.8 to 1.5. The amount of steam in the dehydrogenation reactor effluent can be in the range of from about 0.1 to about 20 moles of steam per mole of styrene, but, more typically, the steam-to-styrene molar ratio is in the range of from 0.8 to 8. In terms of molar percent concentration, the dehydrogenation reactor effluent can have a concentration of hydrogen in the range of from about 30 to about 80 mole percent based on the total moles of the dehydrogenation reactor effluent stream but excluding the steam component, and more specifically, such concentration can be in the range of from 40 to 70 mole percent. The mole percent concentration of styrene in the dehydrogenation reactor effluent can be in the range of from about 30 to about 80 mole percent based on the total moles of the dehydrogenation reactor effluent stream but excluding the steam component, and more specifically, such concentration can be in the range of from 40 to 70 mole percent. The dehydrogenation reactor effluent can also further comprise other compounds such as toluene and benzene.

To selectively oxidize at least a portion of the hydrogen contained in the dehydrogenation reactor effluent to water, the dehydrogenation reactor effluent is introduced along with an oxygen-containing gas into an oxidation reaction zone that is defined by an oxidation reactor vessel and which contains a low temperature selective oxidation catalyst as is more fully described below. The dehydrogenation reactor effluent is thereby contacted under suitable low temperature selective oxidation conditions and in the presence of oxygen with the selective oxidation catalyst to yield a selectively oxidized reaction product having a reduced amount of hydrogen relative to the amount of hydrogen in the dehydrogenation reactor effluent.

The oxygen-containing gas introduced into and admixed with the dehydrogenation reactor effluent can be any suitable gas that provides the oxygen necessary for conducting the low temperature selective oxidation reaction of hydrogen to water. Examples of such suitable gases include air, oxygen and air or oxygen that is diluted with other gases such as steam, carbon dioxide and the inert gases such as nitrogen, argon, and helium. Preferred is a high purity oxygen-containing gas having a high concentration of oxygen, for instance, greater than 90 volume percent, or greater than 95 volume percent or even greater than 98 volume percent oxygen. The amount of oxygen combined with the dehydrogenation reactor effluent is such as to provide a mole ratio of oxygen-to-hydrogen in the range of from about 0.1:1 to about 2:1, but, preferably, the mole ratio of oxygen-to-hydrogen should approach the stoichiometric requirements and, thus, a preferred range for the mole ratio is from 0.2 to 0.8, and most preferred, from 0.3 to 0.7.

An important feature of the invention is that the selective oxidation step is conducted at low temperature reaction conditions as compared to the prior art processes that conduct their selective oxidation step at significantly higher reaction temperatures. Indeed, one significant benefit provided by the inventive method for the processing of a dehydrogenation reactor effluent comprising styrene and hydrogen is the especially high conversion of hydrogen that is achieved but with a simultaneously low conversion of the styrene both of which are achieved at a low selective oxidation reaction temperatures. It is recognized that the particular selective oxidation catalysts described herein provide for the aforementioned selective oxidation of hydrogen that is contained in the dehydrogenation reactor effluent at the low temperature reaction conditions.

The selective oxidation catalyst of the invention is a catalyst composition comprising a noble metal of either platinum or palladium, or both, supported on an inorganic oxide support material and which provides for the effective low temperature selective oxidation of hydrogen when in the presence of an oxidatable hydrocarbon, such as, for example, the selective oxidation of hydrogen contained in a dehydrogenation reactor effluent stream, or a dehydrogenate.

When referring herein to the low temperature of a selective oxidation reaction, what is meant is that the selective oxidation reaction is conducted at a temperature significantly lower than the typical temperatures at which an iron catalyzed ethylbenzene dehydrogenation reaction is conducted. Generally, this is significantly less than about 500° C. It is a recognized aspect of the invention that, for the selective oxidation of hydrogen in an ethylbenzene dehydrogenate comprising styrene and hydrogen, a low temperature for the selective oxidation of the hydrogen is essential in order to minimize the corresponding undesirable conversion of styrene. The low temperature of the selective oxidation reaction of the invention, thus, is less than about 250° C.; and, since it is best for the water component of the dehydrogenate feed to the selective oxidation reactor to be in the vapor state, the temperature of the selective oxidation reaction can be in the range of from about 100° C. to 250° C., and, more specifically, in the range of from 100 to 240° C. It is preferred, however, for the selective oxidation reaction temperature to be in the range of from 100 to 220° C., and, most preferred, from 100 to 200° C. In certain circumstances, an optimum selective oxidation temperature can be less than 180° C.

When referring herein to the selective oxidation reaction, what is meant is that the hydrogen of the selective oxidation reactor feed, such as a dehydrogenation reactor effluent that comprises hydrogen and an oxidizable hydrocarbon, such as styrene, is preferentially converted to water with a relatively smaller conversion of the oxidizable hydrocarbon. The aforedescribed temperature ranges for the selective oxidation reaction are significant in that they are important to maintaining the selectivity of the reaction toward the oxidation of hydrogen.

In the selective oxidation of hydrogen contained in an ethylbenzene dehydrogenate comprising styrene and hydrogen, a selective oxidation reaction is defined as being when more than about 50 mole percent of the hydrogen is converted with less than about 50 mole percent of the styrene being converted. A higher selectivity, however, is more desired in that the hydrocarbon of the ethylbenzene dehydrogenate is a preferred end-product. Thus, the selectivity of the oxidation reaction should be such that the hydrogen conversion exceeds 70 mole percent when the styrene conversion is less than 35 mole percent. Preferred, however, is for the hydrogen conversion to exceed 95 mole percent when the styrene conversion is less than 25 mole percent, and, most preferred, the hydrogen conversion exceeds 97 mole percent when the styrene conversion is less than 15 mole percent, or even, less than 10 mole percent.

The selective oxidation catalyst of the invention provides for the aforedescribed low temperature, high selectivity hydrogen oxidation reaction and, as earlier described herein, the catalyst generally comprises a noble metal of either platinum or palladium, or both, supported on an inorganic oxide support material. The preferred noble metal is platinum. A preferred selective oxidation catalyst, however, further comprises a promoter metal such as tin or rhodium; but the most preferred selective oxidation catalyst comprises both platinum and palladium with a promoter of either tin or rhodium, or both, supported on an inorganic support material. Among the promoters, rhodium is preferred.

The inorganic oxide support material of the selective oxidation catalyst can include alumina that is shaped into any suitable form such as pellets, extrudates, spheres and the like onto which the at least one noble metal and, optionally, promoter metal are incorporated. The inorganic oxide support material can also be of the form of a monolithic carrier made of a refractory material comprised of one or more metal oxides, for example, alumina, alumina-silica, alumina-silica-titania, mullite, cordierite, zirconia, zirconia-spinal, zirconia-mullite, silicon carbide and the like. Preferred among these materials is cordierite, which is an alumina-magnesia-silica material. One catalyst system, which comprises at least one noble metal and, optionally, a promoter metal, supported on a monolithic carrier, suitable for use as a selective oxidation catalyst of the invention is described in detail in U.S. Pat. No. 4,844,837, which is incorporated herein by reference. One commercially available monolithic catalyst system that can suitably be used as a low temperature selective oxidation catalyst system is the palladium on monolith product HEX209 marketed by Engelhard Corporation. This system has relative metal loadings of 1 part rhodium per 40 parts palladium per 1 part platinum with a total metal loading of 105 grams metal per cubic feet of monolith.

The noble metal concentration in the selective oxidation catalyst is such as to provide for the low temperature selective oxidation properties required for the invention and can be in the range of from about 0.005 weight percent to about 5 weight percent of the total weight of the catalyst including the support. It is preferred for the noble metal to be present in the catalyst in the range of from 0.05 to 3 weight percent, and, most preferred, from 0.1 to 0.5 weight percent. As for the promoter metal of the catalyst, it can be present in the range of from about 0.005 weight percent to about 5 weight percent of the total weight of the catalyst including the support. It is preferred for the promoter metal to be present in the catalyst in the range of from 0.05 to 3 weight percent, and, most preferred, from 0.1 to 0.5 weight percent. One commercially available catalyst that can suitably be used as the low temperature selective oxidation catalyst is CRITERION PS-20 marketed by Criterion Catalysts & Technologies. This catalyst has a low platinum and tin concentration of about 0.3 weight percent for each and which are supported on an alumina support.

One embodiment of the invention provides for an improved operation of a dehydrogenation reactor system includes operating, in combination, a dehydrogenation reactor and a low temperature selective oxidation reactor. The equipment arrangement in which the dehydrogenation reactor is connected in fluid flow communication with the selective oxidation reactor and in which the selective oxidation reactor is connected in fluid flow communication with a compressor allows for the reduction in the dehydrogenation reactor pressure to a second dehydrogenation reactor pressure. This pressure reduction may be achieved through the conversion of at least a portion of the hydrogen contained in the dehydrogenation reactor effluent to water, which can then be condensed out of the selectively oxidized reactor effluent prior to passing the remaining vapor phase to the compressor. The resulting reduction in the volume or mass of gas flow to the compressor provides for a significant reduction in back pressure that further provides for a lower operating pressure in the dehydrogenation reactor.

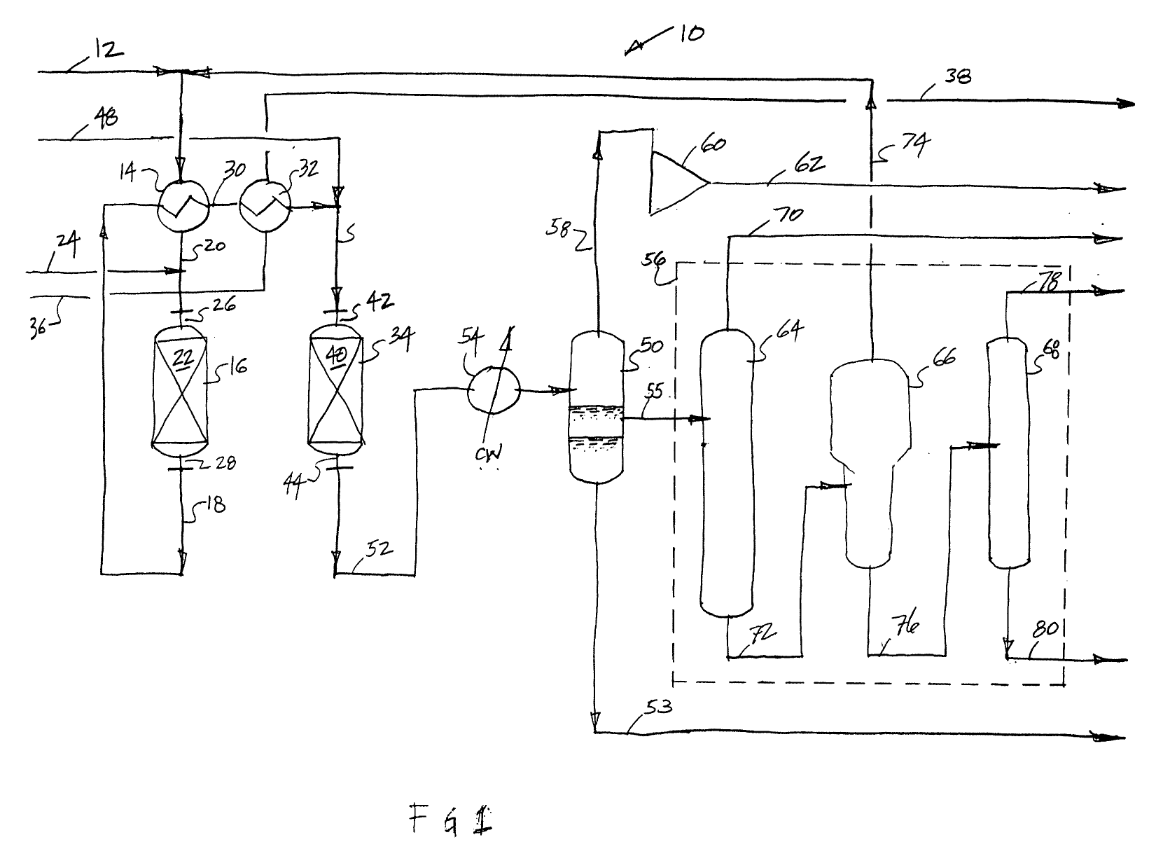

Now referring to FIG. 1, presented is a schematic representation of one possible arrangement of steps of process 10 for the manufacture of styrene by the dehydrogenation of ethylbenzene in which is utilized a step for the low temperature selective oxidation of hydrogen contained in a ethylbenzene dehydrogenate stream, or dehydrogenation reactor effluent.

In process 10, an ethylbezene feed stream, comprising ethylbenzene, passes by way of conduit 12 to feed/effluent heat exchanger 14. Feed/effluent heat exchanger 14 defines a heat transfer zone and provides means for indirect heat exchange with the dehydrogenation reactor effluent passing from dehydrogenation reactor 16 to feed/effluent heat exchanger 14 by way of conduit 18. The heated ethylbenzene feed stream passes from feed/effluent heat exchanger 14 to dehydrogenation reactor 16 through conduit 20. Prior to the introduction of the heated ethylbenzene feed stream into dehydrogenation reactor 16, superheated steam passing by way of conduit 24 is introduced into and admixed with the heated ethylbenzene feed stream to provide additional heat required for the dehydrogenation of ethylbenzene.

Dehydrogenation reactor 16 defines a dehydrogenation reaction zone that contains a bed of dehydrogenation catalyst 22 and provides means for contacting the ethylbenzene feed stream, under suitable dehydrogenation reaction conditions, with dehydrogenation catalyst 22. Dehydrogenation reactor 16 further includes dehydrogenation reactor feed inlet 26 and dehydrogenation reactor effluent outlet 28. Dehydrogenation reactor feed inlet 26 provides means for receiving a dehydrogenation reactor feed, such as the ethylbenzene feed stream, and dehydrogenation reactor effluent outlet 28 provides means for discharging a dehydrogenation reactor effluent, such as an ethylbenzene dehydrogenate.

A cooled dehydrogenation reactor effluent passes from feed/effluent heat exchanger 14 through conduit 30 to heat transfer unit 32 which defines a heat transfer zone and provides means for the transfer of heat from the cooled dehydrogenation reactor effluent to a cooling medium to thereby further cool the dehydrogenation reactor effluent prior to its introduction into selective oxidation reactor 34. The cooling medium passes to heat transfer unit 32 by way of conduit 36 and the heated cooling medium passes from heat transfer unit 32 by way of conduit 38.

Selective oxidation reactor 34 defines an oxidation reaction zone that contains a bed of low temperature selective oxidation catalyst 40 and provides means for contacting the dehydrogenation reactor effluent, under suitable low temperature selective oxidation reaction conditions, with low temperature selective oxidation catalyst 40. Selective oxidation reactor 34 further includes oxidation reactor feed inlet 42 and oxidation reactor effluent outlet 44. Oxidation reactor feed inlet 42 provides means for receiving a dehydrogenation reactor effluent, such as an ethylbenzene dehydrogenate, and oxidation reactor effluent outlet 44 provides means for discharging a selective oxidation reactor effluent.

The further cooled dehydrogenation reactor effluent passes from heat transfer unit 32 through conduit 46 to selective oxidation reactor 34. Prior to the introduction of the further cooled dehydrogenation reactor effluent into the selective oxidation reactor 34, oxygen-containing gas passing by way of conduit 48 is introduced into and admixed with the further cooled dehydrogenation reactor effluent to provide oxygen required for the hydrogen oxidation reaction. The dehydrogenation reactor effluent admixed with oxygen is introduced into selective oxidation reactor 34 through oxidation reactor feed inlet 42 wherein the dehydrogenation reactor effluent admixed with oxygen is contacted with low temperature selective oxidation catalyst 40 under suitable low temperature selective oxidation reaction conditions so as to selectively oxidize at least a portion of the hydrogen contained in the dehydrogenation reactor effluent.

A selectively oxidized reaction product or reactor effluent having a reduced amount of hydrogen relative to the amount of hydrogen in the dehydrogenation reactor effluent is yielded from selective oxidation reactor 34 through oxidation reactor effluent outlet 44 and passes to separator 50 by way of conduit 52. Cooler 54 is interposed in conduit 52. Cooler 54 defines a heat transfer zone and provides means for removing heat energy from the selectively oxidized reactor effluent.

Separator 50 defines a separation zone and provides means for separating the cooled selectively oxidized reactor effluent into a hydrocarbon stream, comprising hydrocarbons, such as styrene and ethylbenzene, a water stream, comprising water, and a vapor stream, comprising hydrogen. The water stream passes from separator 50 through conduit 53. The hydrocarbon stream passes from separator 50 through conduit 55 and is charged to separation system 56. Separation system 56 defines at least one separation zone and provides means for separating dehydrogenated hydrocarbons, such as styrene, from unconverted dehydrogenatable hydrocarbons, such as ethylbenzene, and other hydrocarbons.

The vapor stream passes from separator 50 through conduit 58 and is introduced into the suction inlet of compressor 60, which defines a compression zone and provides means for compressing the vapor stream. The compressed vapor stream is discharged and passes from compressor 60 through conduit 62. The interposition of selective oxidation reactor 34 in fluid flow communication between dehydrogenation reactor 16 and compressor 60 along with its operation under low temperature selective oxidation conditions provides for an improved operation of the dehydrogenation reactor by allowing for a reduction in its operating pressure. This reduction in dehydrogenation reactor operating pressure is achieved by the combination of interposing low temperature selective oxidation reactor 34 between dehydrogenation reactor 16 and compressor 60 and selectively oxidizing a portion of the hydrogen contained in the dehydrogenation reactor effluent to water. The water is then condensed out of the selectively oxidized reactor effluent to thereby reduce the vapor stream volume introduced into the suction of compressor 60. The resulting reduction in back pressure as a result of a lower vapor stream volumetric flow provides for a reduction in the operating pressure of dehydrogenation reactor 16.

Separation system 56 can further include benzene-toluene (BT) column 64, ethylbenzene recycle column 66 and styrene finisher 68. The hydrocarbon stream from separator 50 is fed by way of conduit 55 to benzene-toluene column 64 which defines a separation zone and provides means for separating the hydrocarbon stream into a benzene/toluene stream comprising benzene and toluene and a BT column bottoms stream comprising ethylbenzene and styrene. The benzene/toluene stream passes from BT column 64 through conduit 70.

The BT column bottoms stream passes from BT column 64 through conduit 72 and is charged to ethylbenzene recycle column 66. Ethylbenzene recycle column 66 defines a separation zone and provides means for separating the BT column bottoms stream into an ethylbenzene recycle stream, comprising ethylbenzene, and an ethylbenzene recycle column bottoms stream, comprising styrene. The ethylbenzene recycle stream passes from ethylbenzene recycle column 66 through conduit 74 and is combined with the ethylbenzene feed stream being changed to feed/effluent exchanger 14 via conduit 12. The ethylbenzene recycle column bottoms stream passes from ethylbenzene recycle column 66 through conduit 76 to styrene finisher 68. Styrene finisher 68 defines a separation zone and provides means for separating the ethylbenzene recycle column bottoms stream into a styrene product stream, comprising styrene, and a residue stream. The styrene product stream passes from styrene finisher 68 through conduit 78 and the residue stream passes through conduit 80.

The following example is presented to further illustrate the invention, but it is not to be construed as limiting the scope of the invention.

### EXAMPLE

This Example presents the results from testing the performance of six different catalyst systems for their performance in the selective oxidation of hydrogen that is contained in a simulated ethylbenzene dehydrogenate stream that comprises hydrogen, styrene and water.

The following six catalysts were tested for their low temperature selective oxidation performance:

A. Engelhard Corporation palladium monolith HEX209 having a total metal loading of 105 grams per cubic feet of monolith with the relative weight loadings of 1 part rhodium per 40 parts palladium per 1 part platinum;

B. Johnson Matthey platinum on cordierite monolith;

C. CRITERION PS-20 low platinum (0.3 wt. %) and tin (0.3 wt. %) supported on alumina;

D. Five weight percent (5%) platinum supported on alpha alumina;

E. Five weight percent (5%) platinum supported on yiftria stabilized zirconia foam; and

F. Five weight percent (5%) rhodium supported on alpha alumina.

A sample of approximately 120 mg of each of the six catalyst systems listed above was placed in a laboratory nanoreactor. For the two monolith catalyst systems A and B, a small section of the monolith was used. For the other catalyst systems C, D, E, and F, the catalyst was crushed into 30 to 40 mesh (approximately 0.4 mm) particles that were used to pack the nanoreactor.

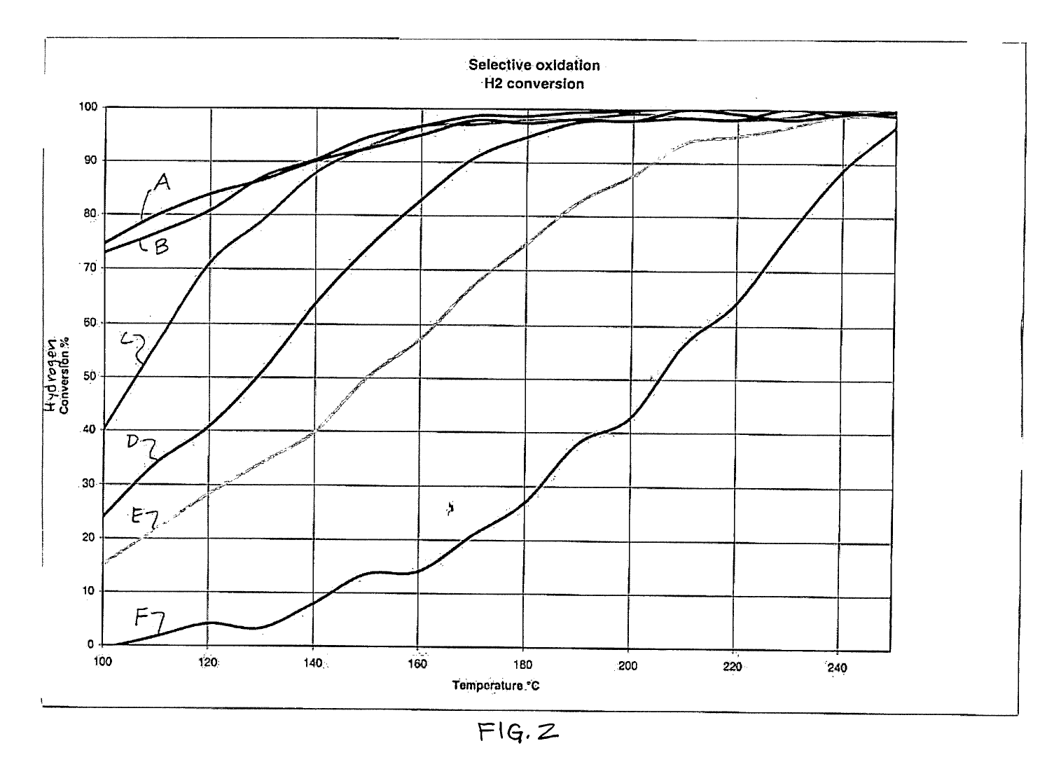

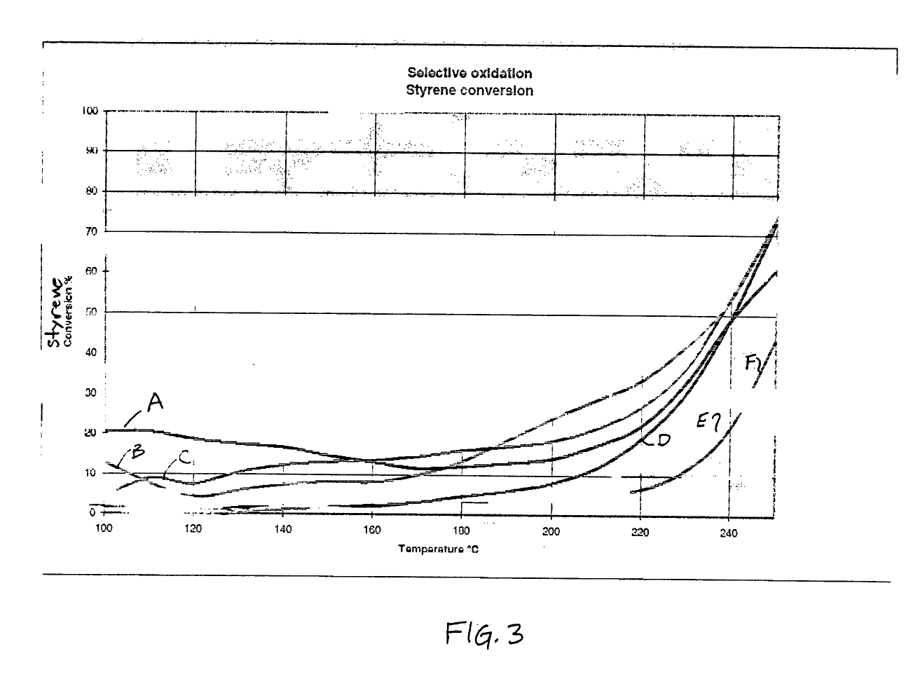

A simulated dehydrogenation reactor effluent gas stream additionally containing oxygen was passed over the catalyst of each reactor at a rate such that the gaseous hourly space velocity was 50,000 hr⁻¹. The reactor was operated at a pressure of about 1 bar. The simulated dehydrogenation reactor effluent included 1700 ppm styrene, 1500 ppm hydrogen, 10,000 ppm water and 10,000 ppm oxygen. The temperature of the reactor was raised in 5° C. increments starting at an initial temperature of 100° C. After the reactor temperature became stable, the composition of the reactor effluent was determine by using an online mass spectrometer. The percent conversion of hydrogen and the percent conversion of styrene results from the above testing are presented in FIG. 2 and FIG. 3.

FIG. 2 presents plots of the percent hydrogen conversion as a function of reaction temperature for each of the catalyst systems tested. As can be observed from these plots, for the very low reaction temperatures, e.g. below about 140° C. to 150° C. or 180° C., the monolith type catalyst systems A and B provided for the highest percent hydrogen conversion. The hydrogen conversion for catalyst systems A and B in all cases exceeded 70 percent, and for temperatures exceeding 140° C. the percent hydrogen conversion exceeded 90 percent. The hydrogen conversion for the catalyst systems A and B exceeded 97 percent for reaction temperatures exceeding 160 to 180° C. Catalyst system C. provided hydrogen conversions comparable to those for catalyst systems A and B at reaction temperatures exceeding 140° C., but it did not provide for as high a percent hydrogen conversion at the lower reaction temperatures. It is noted that the monolithic type catalyst systems and the promoted platinum catalyst systems performed best, and the non-platinum containing rhodium supported on alumina catalyst system F provided for the lowest percent hydrogen conversion.

FIG. 3 presents plots of the percent styrene conversion as a function of reaction temperature for each of the catalyst systems tested. It is noted that for all the catalyst systems A through F provided for percent styrene conversions that were below about 20 to 15 percent and even less than 10 percent at reaction temperatures below about 180 to 185° C.

Reasonable variations, modifications and adaptations can be made within the scope of the described disclosure and the appended claims without departing from the scope of the invention.

That which is claimed is:

1. A method for the low temperature selective oxidation of hydrogen contained in a reactor effluent of a dehydrogenation reactor, said method comprises:
adding an oxygen containing gas to said reactor effluent thereby forming a selective oxidation reaction gas;
contacting under low temperature selective oxidation conditions said selective oxidation reaction gas with a selective oxidation catalyst that is effective in the selective oxidation of hydrogen contained in said selective oxidation reaction gas; and
yielding a selectively oxidized reaction product having a reduced amount of hydrogen relative to the amount of said hydrogen in said reactor effluent.

2. A method as recited in claim 1, wherein said reactor effluent is made by the dehydrogenation of ethylbenzene to yield styrene, and wherein said reactor effluent comprises hydrogen and styrene.

3. A method as recited in claim 2, wherein said selective oxidation catalyst comprises a noble metal supported on an inorganic oxide support.

4. A method as recited in claim 3, wherein said selective oxidation catalyst further comprises an additional metal selected from tin, rhenium or rhodium.

5. A method as recited in claim 4, wherein the amount of noble metal in said selective oxidation catalyst is in the range of from about 0.1 weight percent to about 10 weight percent.

6. A method as recited in claim 5, wherein said low temperature selective oxidation conditions include a selective oxidation temperature in the range upwardly to about 250° C.

7. A method as recited in claim 6, wherein the amount of hydrogen of said selective oxidation reaction gas converted is in the range exceeding about 40 weight percent of the total hydrogen in said selective oxidation reaction gas.

8. A method as recited in claim 7, wherein the amount of styrene of said selective oxidation reaction gas converted is less than about 50 weight percent of the total styrene in said selective oxidation reaction gas.

9. A method as recited in claim 8, wherein said low temperature selective oxidation conditions are such as to simultaneously provide for a high hydrogen conversion and a low styrene conversion.

10. A method for the low temperature selective oxidation of hydrogen contained in a reactor effluent of a dehydrogenation reactor, said method comprises:
selectively oxidizing at least a portion of said hydrogen contained in said reactor effluent by contacting said reactor effluent under low temperature selective oxidation conditions and in the presence of oxygen with a selective oxidation catalyst that is effective in the selective oxidation of hydrogen when in the presence of an oxidatable hydrocarbon.

11. A method as recited in claim 10, wherein said reactor effluent is made by the dehydrogenation of ethylbenzene to yield styrene, and wherein said reactor effluent comprises hydrogen and styrene.

12. A method as recited in claim 11, wherein said selective oxidation catalyst comprises a noble metal supported on an inorganic oxide support.

13. A method as recited in claim 12, wherein said selective oxidation catalyst further comprises an additional metal selected from tin, rhenium or rhodium.

14. A method as recited in claim 13, wherein the amount of noble metal in said selective oxidation catalyst is in the range of from about 0.1 weight percent to about 10 weight percent.

15. A method as recited in claim 14, wherein said low temperature selective oxidation conditions include a selective oxidation temperature in the range upwardly to about 250° C.

16. A method as recited in claim 15 wherein the amount of hydrogen contained in said reactor effluent converted is in the range exceeding about 40 weight percent of the total hydrogen in said reactor effluent.

17. A method as recited in claim 16 wherein the amount of styrene of said reactor effluent converted is less than about 50 weight percent of the total styrene in said reactor effluent.

18. A method as recited in claim 17, wherein said low temperature selective oxidation conditions are such as to simultaneously provide for a high hydrogen conversion and a low styrene conversion.

19. A method of improving the operation of a dehydrogenation reactor system operated under dehydrogenation reaction conditions including a first dehydrogenation pressure wherein yielded from said dehydrogenation reactor system is a reactor effluent containing hydrogen, said method comprises the steps of:
providing a low temperature selective oxidation reactor system operatively connected with said dehydrogenation reactor system so as to be capable of receiving as a feed said reactor effluent; and
operating said low temperature selective oxidation reactor system under low temperature selective oxidation conditions so as to selectively oxidize at least a portion of said hydrogen contained in said reactor effluent and to reduce said dehydrogenation pressure of said dehydrogenation reactor system.

20. A method as recited in claim 19, wherein said reactor effluent is made by the dehydrogenation of ethylbenzene to yield styrene, and wherein said reactor effluent comprises hydrogen and styrene.

21. A method as recited in claim 20, wherein said low temperature selective oxidation reactor system comprises an oxidation reactor which defines an oxidation reaction zone containing a low temperature selective oxidation catalyst and includes an oxidation reactor feed inlet for receiving said reactor effluent and an oxidation reactor effluent outlet for discharging a selectively oxidized reactor effluent.

22. A method as recited in claim 20, wherein said low temperature selective oxidation catalyst comprises a noble metal supported on an inorganic oxide support.

23. A method as recited in claim 22, wherein said low temperature selective oxidation catalyst further comprises an additional metal selected from tin, rhenium or rhodium.

24. A method as recited in claim 23, wherein the amount of noble metal in said low temperature selective oxidation catalyst is in the range of from about 0.1 weight percent to about 10 weight percent.

25. A method as recited in claim 24, wherein said low temperature selective oxidation conditions include a selective oxidation temperature in the range upwardly to about 250° C.

26. A method as recited in claim 25, wherein the amount of hydrogen of said reactor effluent converted is in the range exceeding about 40 weight percent of the total hydrogen in said reactor effluent.

27. A method as recited in claim 26, wherein the amount of styrene of said reactor effluent converted is less than about 50 weight percent of the total styrene in said reactor effluent.

28. A method as recited in claim 27, wherein said low temperature selective oxidation conditions are such as to simultaneously provide for a high hydrogen conversion and a low styrene conversion.

29. A method as recited in claim 28, further comprising:
operating said dehydrogenation reactor system at a second dehydrogenation pressure that is lower than said first dehydrogenation pressure.

30. A method, comprising:
providing a dehydrogenation reactor system comprising a dehydrogenation reactor which defines a dehydrogenation reaction zone containing a dehydrogenation catalyst and includes a dehydrogenation reactor feed inlet for receiving a dehydrogenation reactor feed and a dehydrogenation reactor effluent outlet for discharging a dehydrogenation reactor effluent;
providing a low temperature selective oxidation reactor system comprising an oxidation reactor which defines an oxidation reaction zone containing a low temperature selective oxidation catalyst and includes an oxidation reactor feed inlet for receiving said dehydrogenation reactor effluent and an oxidation reactor effluent outlet for discharging a selectively oxidized reactor effluent;
operating said dehydrogenation reactor system under dehydrogenation reaction conditions including a first dehydrogenation pressure so as to yield from said dehydrogenation reaction zone said dehydrogenation reactor effluent containing hydrogen;
introducing said dehydrogenation reactor effluent into said oxidation reaction zone through said oxidation reactor feed inlet while operating said oxidation
reaction zone under low temperature selective oxidation reaction conditions so as to selectively oxidize at least a portion of said hydrogen contained in said reactor effluent and to reduce said first dehydrogenation pressure of said dehydrogenation reactor system to a second dehydrogenation pressure; and
yielding from said oxidation reaction zone said selectively oxidized reactor effluent through said oxidation reactor effluent outlet.

31. A method as recited in claim 30, wherein said reactor effluent is made by the dehydrogenation of ethylbenzene to yield styrene, and wherein said reactor effluent comprises hydrogen and styrene.

32. A method as recited in claim 31, wherein said low temperature selective oxidation reactor system comprises a noble metal supported on an inorganic oxide support.

33. A method as recited in claim 32, wherein said low temperature selective oxidation catalyst comprises a noble metal supported on an inorganic oxide support.

34. A method as recited in claim 33, wherein said low temperature selective oxidation catalyst further comprises an additional metal selected from tin, rhenium or rhodium.

35. A method as recited in claim 34, wherein the amount of noble metal in said low temperature selective oxidation catalyst is in the range of from about 0.1 weight percent to about 10 weight percent.

36. A method as recited in claim 35, wherein said low temperature selective oxidation conditions include a selective oxidation temperature in the range upwardly to about 250° C.

37. A method as recited in claim 36 wherein the amount of hydrogen of said reactor effluent converted is in the range exceeding about 40 weight percent of the total hydrogen in said reactor effluent.

38. A method as recited in claim 37, wherein the amount of styrene of said reactor effluent converted is less than about 50 weight percent of the total styrene in said reactor effluent.

39. A method as recited in claim 27, wherein said low temperature selective oxidation conditions are such as to simultaneously provide for a high hydrogen conversion and a low styrene conversion.

40. A method as recited in claim 39, further comprising:
operating said dehydrogenation reactor system at a second dehydrogenation pressure that is lower than said first dehydrogenation pressure.
